(19) United States
(12) Patent Application Publication
(10) Pub. No.: US 2017/0209195 A1
(43) Pub. Date: Jul. 27, 2017

(54) IMPLANT AND SURGICAL KIT FOR HOLDING BONE BODIES OF A PATIENT IN POSITION WITH RESPECT TO ONE ANOTHER

(71) Applicant: IN2BONES, Ecully (FR)

(72) Inventors: Grégoire Chick, Geneva (CH); Michaël Papaloïzos, Geneva (CH)

(73) Assignee: IN2BONES, Ecully (FR)

(21) Appl. No.: 15/325,017

(22) PCT Filed: Jul. 9, 2015
(86) PCT No.: PCT/FR2015/051904

§ 371 Date: Jan. 9, 2017

(30) Foreign Application Priority Data
Jul. 10, 2014 (FR) 14 56664

(51) Int. Cl. A61B 17/80; A61B 17/17

(52) U.S. Cl. CPC A61B 17/8052; A61B 17/8061; A61B 17/1728

(57) ABSTRACT

The invention concerns a surgical implant (1) intended to be attached on bone bodies (2, 3) of a patient, via fastening means (4, 5, 6, 7) in order to hold in position said bone bodies (2, 3) relative to each other, said surgical implant (1) including a main body (13) formed by a distal plate (11) which is extended by a proximal tab (12), the distal plate (11) extending substantially according to a distal extension plane (Pd), the proximal tab (12) extending substantially along a proximal extension plane (Pp) intersecting the distal extension plane (Pd), the proximal tab (12) and the distal plate (11) being linked by a bending area (19), the surgical implant (1) being characterized in that the bending area (19) has the highest thickness of the main body (13).

### TECHNICAL FIELD

The invention relates to the general field of the surgical implants for osteosynthesis, in particular designed for the treatment of fractures or cracks of bone bodies located in particular in the forearm.

The invention concerns more particularly a surgical implant intended to be attached on bone bodies of a patient, for example his forearm, via fastening means in order to hold in position said bone bodies relative to each other, said surgical implant including a main body formed by a distal plate which is extended by a proximal tab, the distal plate extending substantially according to a distal extension plane, the proximal tab extending substantially along a proximal extension plane intersecting the distal extension plane, the proximal tab and the distal plate being linked by a bending area.

The invention also concerns a surgical kit including a surgical implant.

### PRIOR ART

In order to improve the reconstruction of the forearm bones of a patient as a result of some types of fractures or cracks of the distal end of the radius, it is possible to resort to a surgical implant, generally designated by the term <<radial plate>>. This type of surgical implant is most often formed by a bent metal plate so that the anterior surface of the plate has a convex shape to conform to the shape of the extreme portion of the radius to be repaired. The radial plate is intended to be fastened by screwing on two parts of the radius separated by fracture so that said plate spans the fracture line and immobilizes the two parts of the radius relative to each other to promote their fusion.

If this solution is generally satisfactory and allows the osteosynthesis of the fractured or cracked radius of the patient, it seemed however that it could be improved.

Indeed, due to the very great variety of morphologies of the radius of the patients, the latter are likely, in some cases, to experience pain during the osteosynthesis process. Sometimes, the friction of tissues on the implant and in particular on the ridges of the latter, can also be a source of pain, despite the small thickness of the latter.

It also seems that the speed of the relatively long bone reconstruction process could be improved, in so far as it generally involves an immobilization and/or a protection of the wrist, which is generally uncomfortable and disturbing for the patient.

### DISCLOSURE OF THE INVENTION

The objects assigned to the invention aim consequently to overcome the aforementioned drawbacks, by providing a new surgical implant and a new associated surgical kit allowing to accelerate the process of the osteosynthesis and the recovery of the patient, while being perfectly tolerated.

Another object of the invention aims to provide a new surgical implant and a new associated surgical kit allowing to obtain a good quality osteosynthesis.

Another object of the invention aims to provide a new surgical implant and a new associated surgical kit allowing to limit the pain experienced by the patient.

Another object of the invention aims to provide a new surgical implant and a new associated surgical kit allowing to facilitate the insertion operation of the implant in the patient's body.

Another object of the invention aims to provide a new surgical implant and a particularly polyvalent new associated surgical kit adapting to any type of morphology.

Another object of the invention aims to provide a new surgical implant and a new associated surgical kit whose design and manufacture are relatively easy.

Another object of the invention aims to provide a new surgical implant and a new associated surgical kit whose setting up in the patient's body is facilitated and accurate.

The objects assigned to the invention are achieved with the help of a surgical implant intended to be attached on bone bodies of a patient, for example, his forearm, via fastening means, in order to hold in position said bone bodies relative to each other, said surgical implant including a main body formed by a distal plate which is extended by a proximal tab, the distal plate extending substantially according to a distal extension plane, the proximal tab extending substantially along a proximal extension plane intersecting the distal extension plane, the proximal tab and the distal plate being linked by a bending area, the surgical implant being characterized in that the bending area has the highest thickness in the main body.

The objects assigned to the invention are also achieved with a surgical kit including a surgical implant according to the invention, and being characterized in that it also includes a shoe complementary in shape to the distal plate, so as to be removably attachable on said distal plate, for example during the setting up of the surgical implant in a patient's body.

### BRIEF DESCRIPTION OF THE DRAWINGS

Other features and advantages of the invention will appear and emerge in more detail upon reading the description hereinafter, with reference to the annexed drawings, given only for illustrative and non-restrictive purposes, in which:

FIG. 1 illustrates, according to a general perspective view, a surgical implant according to the invention.

FIG. 2 illustrates, according to a perspective view, the surgical implant of FIG. 1, the view being oriented so that a posterior surface of the latter is shown in the foreground.

FIG. 3 shows, according to a perspective view, the surgical implant of FIGS. 1 and 2, the view being oriented so that a distal edge of the latter is shown in the foreground.

FIGS. 4 and 5 illustrate, according to partial perspective views, a detail of embodiment of the surgical implant of FIGS. 1 to 3, the views being oriented so that a portion of the posterior surface and the distal edge are particularly visible.

FIGS. 6 and 7 illustrate, according to partial perspective views, a detail of embodiment of the surgical implant of FIGS. 1 to 5, the views being oriented so that a portion of the anterior surface and the distal edge are particularly visible.

FIGS. 8 and 9 illustrate respectively, according to perspective views, a fastening screw of the surgical implant of FIGS. 1 to 7 whose head includes retention means, and a fastening screw of said surgical implant whose head is devoid of retention means.

FIGS. 10 and 11 show respectively according to a side view and a top view, the surgical implant of FIGS. 1 to 7 fastened to bone bodies of a patient by means of the fastening screws of FIGS. 8 and 9.

FIG. 12 shows according to an exploded perspective view, a surgical kit according to the invention including the surgical implant of FIGS. 1 to 7 and 10 and 11, as well as a shoe.

FIG. 13 illustrates, according to a cross-sectional view of the surgical implant of the previous figures, an example of embodiment of a screw opening forming a fastening means of said surgical implant.

### BEST WAY TO REALIZE THE INVENTION

The previously mentioned Figures are advantageously shown to scale, in an illustrative and non-restrictive aim of a preferable variant of the invention.

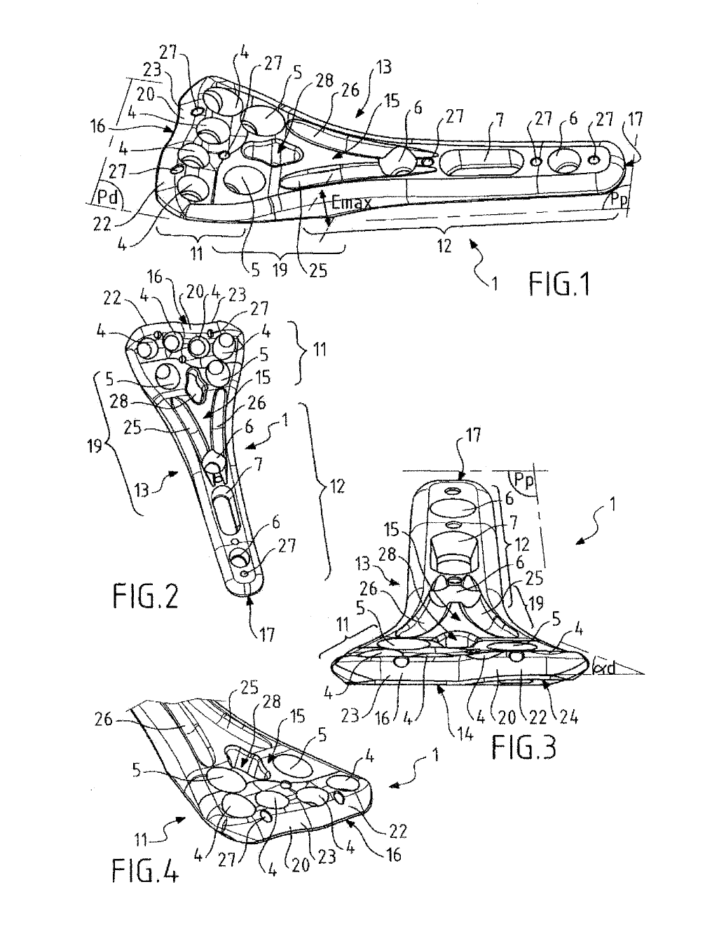

FIGS. 1 to 8 illustrate to a preferable variant of a surgical implant 1 in accordance with the invention. The surgical implant 1, in its preferred variant as shown in Figures, forms a forearm implant, in this case a radial plate for a fracture of the distal end of the radius of a patient's forearm. The invention is however not limited to a forearm implant and will be able to concern for example a tibial implant or a palmar implant. In any event, the surgical implant 1 is provided to be implanted in the patient's body during a surgical operation carried out, for example, under anesthesia, in particular as a result of a lesion of a patient's bone of the type fracture or bone crack, in order to cure or contribute to cure said lesion during an osteosynthesis process. The patient designated by the invention is preferably a human being, on the understanding that there is no impediment to the introduction of the implant 1 of the invention in an animal's body for curing veterinary pathologies similar to human pathologies described herein.

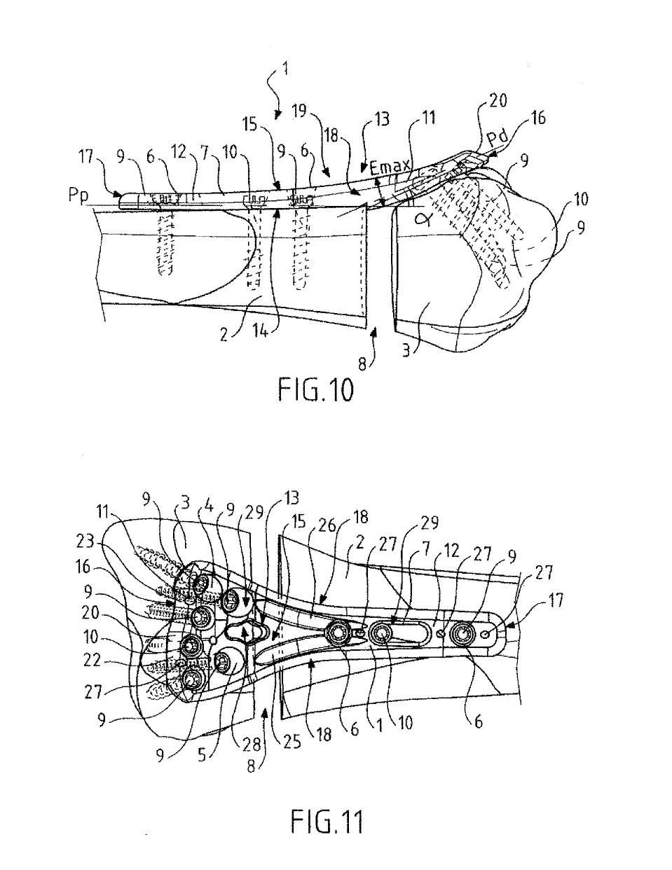

The surgical implant 1 of the invention is intended to be attached on bone bodies 2, 3 of a patient, for example his forearm and/or his wrist, by means of fastening means 4, 5, 6, 7 in order to hold in position said bone bodies 2, 3 relative to each other, such as illustrated for example in FIGS. 10 and 11, particularly to allow, or at least to promote, the osteosynthesis of the latter.

The surgical implant 1 is thus designed to be introduced in a patient's body, and to be fastened temporarily or permanently to the injured bone bodies 2, 3. It is meant by <<fastening means>> elements belonging or not to the surgical implant 1 and allowing to secure the latter to the bone bodies 2, 3. In this case, the surgical implant 1 includes a main body 13, which is intended to be attached on the bone bodies 2, 3. The fastening means 4, 5, 6, 7 are preferably formed by screw openings 4, 5, 6, 7, in which fastening screws 9, 10 are intended to be inserted, in order to secure said surgical implant 1, in particular the main body 13 thereof, with the bone bodies 2, 3 by screwing said fastening screws 9, 10 in the latter, as described below. Other fastening means can be considered without departing from the scope of the invention, such as for example clipping, stapling, nailing means, or the like.

It is meant by bone bodies 2, 3 bones, cartilages, bone or cartilage fragments, or even tendons, or a combination of the latter, preferably intended to be fused by osteosynthesis by means of the surgical implant 1. The surgical implant 1 being preferably a radial plate, the bone bodies 2, 3 are preferably formed by a bone of the cracked or fractured radius of the patient's forearm. In particular, the surgical implant 1 is intended to be set up on the ventral part of the forearm, that is to say the part of the forearm in the extension of the palm of the hand. Of course, the surgical implant 1 of the invention can be set up on other bone bodies of the patient for curing other types of lesions without departing from the scope of the invention.

The setting up of the surgical implant 1 on the bone bodies 2, 3 destined to substantially immobilize the latter relative to each other. Indeed, the surgical implant 1 is intended to be fastened at least to a first and a second bone bodies 2, 3, to link said bone bodies 2, 3 to each other by forming a bridge capable of holding in relative position said bone bodies 2, 3 by spanning for example a fracture line 8, or more generally a separation line of the bone bodies 2, 3. The weakened or cracked bone can thus be advantageously reconstructed, its bone fragments formed by the bone bodies 2, 3 being held in position by the surgical implant 1 in an adequate position for osteosynthesis.

Of course, it may be considered to use such a surgical implant 1 for holding or contributing to hold in position a higher number of bone bodies relative to each other, for example three, four, or more.

According to the invention, the surgical implant 1 includes a main body 13. The latter, and more generally the surgical implant 1, preferably forms a one piece and unitary part. Of course, the surgical implant 1, and particularly its main body 13, can be, on the contrary, the result of the assembly of several parts.

This one piece part is preferably made of a radiolucent material, preferably polyether ether ketone (PEEK), the latter being optionally loaded with carbon, which leads to a good compromise between flexibility and rigidity of the main body capable of promoting the osteosynthesis and a rapid recovery of the bone lesions. Alternatively, the surgical implant 1 can be made of a metal material such as the titanium. The surgical implant 1 can be carried out in one piece by a molding operation, optionally followed by a machining operation of the molded blank, so that it is easy to manufacture.

The main body 13 advantageously has an anterior surface 14 intended to rest at least partially on said bone bodies 2, 3, and a posterior surface 15 opposite to the anterior surface 14 forming a preferably free dorsal surface when the surgical implant 1 is in place on said bone bodies 2, 3.

According to the invention, the main body 13 is formed by a distal plate 11 which is extended by a proximal tab 12, as illustrated in FIGS. 1 to 8. The distal plate 11 forms a portion of the main body 13 advantageously located at a first end of the latter, the proximal tab 12 forming the other end in the continuity of said distal plate 11. The main body 13 extends preferably longitudinally between:

a distal edge 16, from which extends the distal plate 11, and

a proximal edge 17, from which extends the proximal tab 12, and transversely between two side edges 18.

In the sense of the invention, the longitudinal direction of the surgical implant 1 extends from the distal edge 16 to the proximal edge 17, whereas the transverse direction extends from one side edge 18 to the other. Finally, the sagittal direction shows the third spatial direction orthogonal both to the longitudinal and the transverse directions.

The anterior surface 14 preferably extends from the distal plate 11 to the proximal tab 12, continuously, smoothly and longitudinally, on an anterior side of the latter. Similarly, the posterior surface 15 preferably extends from the distal plate 11 to the proximal tab 12, continuously, smoothly and longitudinally, on an anterior side of the latter.

The distal plate 11 is advantageously designed and shaped to be attached to a preferably distal extreme bone body, that is to say located at the front or the anterior of the bone bodies 2, 3, preferably at a distal part of the bone of the radius, that is to say closest to the wrist of the patient. The distal plate 11 of the invention extends substantially according to a distal extension plane Pd, forms a flattened portion of the main body 13, and has preferably the shape of a spatula. The distal plate 11 is provided to be fastened flat on the bone body 3 resting on the anterior surface 14 (as illustrated in FIGS. 10 and 11). The distal extension plane Pd extends in the transverse direction, and in a direction close to the longitudinal direction.

The proximal tab 12, unlike the distal plate 11, is advantageously tapered, in the shape of a rod or a spatula handle, and is transversely thinner than the latter. The proximal tab 12 is advantageously straight, but can be transversely bent. Indeed, the proximal tab 12 is preferably intended to be attached on:

a median bone body, distant from the joints or the ends of the considered bone, and/or

a proximal bone body, close to a bone end opposite to the distal extreme bone body.

In particular, the proximal tab 12 can be designed to be attached on a median part of the bone of the radius of the patient. The proximal tab 12, if it is thinner than the distal plate 11, is however advantageously slightly flattened to form the anterior surface 14, it is intended to be attached on the bone body 2. According to the invention, the proximal tab 12 extends substantially along a proximal extension plane Pp, according to the invention. The proximal extension plane Pp extends in the transverse direction, and in a direction close to the longitudinal direction.

According to the invention, the proximal extension plane Pp intersecting the distal extension plane Pd, the proximal tab 12 and the distal plate 11 being linked by a bending area 19. The main body 13, according to the invention, is thus slightly flexed in the longitudinal direction, preferably about a transverse axis, in order to adapt to the bone morphology of the bone bodies 2, 3, in particular a bone of the radius. The proximal extension plane Pp and the distal extension plane Pd are distinct and thus form a slight angle about the transversal direction, which is particularly shown in FIG. 10. The distal plate 11 is thus slightly raised relative to the proximal tab 12. The main body 13, from one end to the other, thus includes three sections, namely the proximal tab 12 followed by the bending area 19, itself followed by the distal plate 11. Preferably, the distal plate 11 is inclined relative to the proximal tab 12, so that the distal extension plane Pd and the proximal extension plane Pp are intersecting each other so as to form an angle of elevation α being comprised between 10° and 80°, preferably between 20° and 30°, and even more preferably about 25°, in order to adapt to the morphology of the bone bodies 2, 3, and in particular to the inclination of the surface of the bone at the end of the latter relative to the surface of the bone in the median part of the latter.

In other words, the angle of elevation α is comparable to an angle of incidence of the distal plate 11 relative to the proximal tab 12. In this case, the angle of elevation α shows the angular deviation between a distal virtual extension of the proximal extension plane Pp, and the distal extension plane Pd. The angle of elevation α reflects the slight terminal camber of the surgical implant 1 at its bending area 19. The angle of elevation α is defined as shown for example in FIG. 10, under the distal plate 11.

In the preferable example illustrated in the figures, the bending area 19 is formed by the combination of the distal end of the proximal tab 12 and the proximal end of the distal plate 11, and forms a curved junction therebetween. The main body 13 is advantageously bent in one direction, in this case the longitudinal direction. Without departing from the scope of the invention, the main body 13 can have other bends to adapt to the morphology of the bone bodies 2, 3, like for example a longitudinal torsion, or a flexion about the longitudinal axis.

In any event, the surgical implant 1, and in particular the main body 13, has a shape which is particularly adapted to the morphology of the bone bodies 2, 3 in order to limit the pain experienced by the patient when the implant is in place.

According to such design, the anterior surface 14 has preferably a convex general shape particularly adapted to the bend and to the shape of the surface of the bone bodies 2, 3, and the posterior surface 15 has a concave general shape.

According to an important characteristic of the invention, the bending area 19 has the highest thickness of the main body 13. In the sense of the invention, it is meant by “thickness” the dimension of the main body 13 in the sagittal direction, that is to say, in the vicinity of the orthogonal to the distal extension plane Pd. In the sense of the invention, the term “thickness”, when applied to the main body 13 as such, does not take account of the orifices, openings, windows, concavities, furrows and the like, arranged in said main body 13, unless otherwise indicated.

According to such design, the rigidity of the surgical implant 1 of the invention is greater at the bending area 19 than at the distal edge 16 and/or the proximal edge 17.

Thus, the surgical implant 1 has a sufficient flexibility to limit the pain experienced by the patient at the fastening means 4, 5, 6, 7, and to stimulate the osteosynthesis by authorizing a slight movement of the bone bodies 2, 3 relative to each other. The flexibility of the surgical implant 1 also allows it to adapt to various morphologies of bone bodies.

The surgical implant 1 of the invention also has a sufficient rigidity to efficiently hold and immobilize in position the bone bodies 2, 3 relative to each other in order to promote reconstruction according to a defined positioning, advantageously corresponding to the position of the bone bodies before fracture or crack. Generally, the surgical implant 1 of the invention allows improving the speed of the recovery and the reconstruction of the patient's bone.

The bending area 19 has advantageously a thickness greater than the thickness of at least one of the distal plate 11 and of the proximal tab 12. Still more preferably, the bending area 19 has a thickness greater than the respective thicknesses of the distal plate 11 and of the proximal tab 12.

In the preferred variant shown in the figures, the bending area 19 includes a maximum thickness Emax, the thickness of the bending area 19 being decreasing from the maximum thickness Emax, on the one hand, in the direction of the distal plate 11, and on the other hand, in the direction of the proximal tab 12.

Preferably, the anterior surface 14 has a bending which is less significant than the posterior surface 15 so as to form a maximum thickness Emax between the proximal tab 12 and the distal plate 11, at the bending area 19.

At least a part of the proximal tab 12 has a constant thickness from the proximal edge 17. Similarly, at least a part of the distal plate 11 has a preferably constant thickness from the distal edge 16. It is also possible to consider that the thickness of the main body 13 is higher in the vicinity of the distal edge 16 and/or of the proximal edge 17 than in the areas located between the maximum thickness Emax and said vicinities (not shown).

In any event, the bending area 19 and in particular the maximum thickness Emax are intended to be placed at the fracture line 8 as illustrated in FIGS. 10 and 11, in order to present maximum rigidity at said fracture line 8 to ensure a reliable holding in position of the bone bodies 2, 3.

Preferably, a through window 28 is arranged through the main body 13 at the bending area 19, in order to allow in particular the insertion of an osteoinductive material in the patient's body, and in particular at the fracture line 8, through said bending area 19 of the surgical implant 1, for example with the aim of repairing a fracture or a crack of the bone bodies 2, 3. The through window 28 is advantageously intended to be placed perpendicularly to the fracture line 8.

Preferably, the side edges 18 are substantially symmetrical relative to each other vis-à-vis a median plane of the main body 13. According to this preferable configuration, the main body 13 has itself a substantially symmetrical general shape. Of course, without departing from the scope of the invention, it can be considered on the contrary that the main body 13 is asymmetrical, and forms, for example, a second bend about the sagittal direction.

As illustrated in Figures, the side edges 18 are preferably curved and diverge from each other from the free end of the proximal tab 12 to the opposite free end of the distal plate 11, so that the proximal tab 12 is extended by the distal plate 11 by widening gradually. The surgical implant thus advantageously has the general shape of a “Y”.

In the variant of the invention shown in Figures, and in particular in FIGS. 3 to 5 and 10, said distal edge 16 is advantageously beveled on the side of the posterior surface 15 so as to have a posterior fillet 20. The presence of this posterior fillet 20 has the dual function:

to provide flexibility to the distal edge 16 suitable for reducing the pain experienced by the patient on the corresponding bone body 3, preferably the distal end of the radius, and

to provide to the distal edge 16 a shape which follows the external extreme bend of the bone body 3, so as to create a continuity in shape likely to limit the risk of abrasion or cut of the patient's body by the distal edge 16, for example the tissues of the wrist of the patient, the tendons, or the nerves.

Preferably, at least one portion of the side edges 18 is beveled on the side of the anterior surface 14 from the distal edge 16, so as to form anterior fillets 21. The presence of the anterior fillets 21 allows the distal plate 11 to conform substantially to the shape of the bone body 3, advantageously the extreme portion of the radius, in order to reduce the pain experienced by the patient and to adapt to varied bone morphologies.

With the aim of adapting finely to the bone morphology of the patient, and in particular of the extreme part of the bone and of the bone bodies 2, 3, the distal edge 16 advantageously has an ulnar portion 22 and a radial portion 23, the latter being recessed relative to said ulnar portion 22 so as to conform to the anatomical line of the bone bodies 2, 3, in particular a bone of the radius of the patient. Preferably, the distal edge 16 extends transversely and is composed of two successive portions, the ulnar portion 22 advanced relative to the radial portion 23. The distal edge 16 thus has advantageously an “S” shape to limit the pain experienced by the patient. It is meant by “ulnar portion” the part of the distal edge 16 intended to be disposed preferably on the ulnar side of the forearm, that is to say substantially in the extension of the little finger. The radial portion is preferably intended to be disposed on the radial side of the forearm, opposite the ulnar side, the radial side being substantially in the extension of the thumb.

Thus, the surgical implant 1has the shape of two main variants formed by an implant for left forearm (not shown in the figures) and an implant for right forearm (shown in the figures).

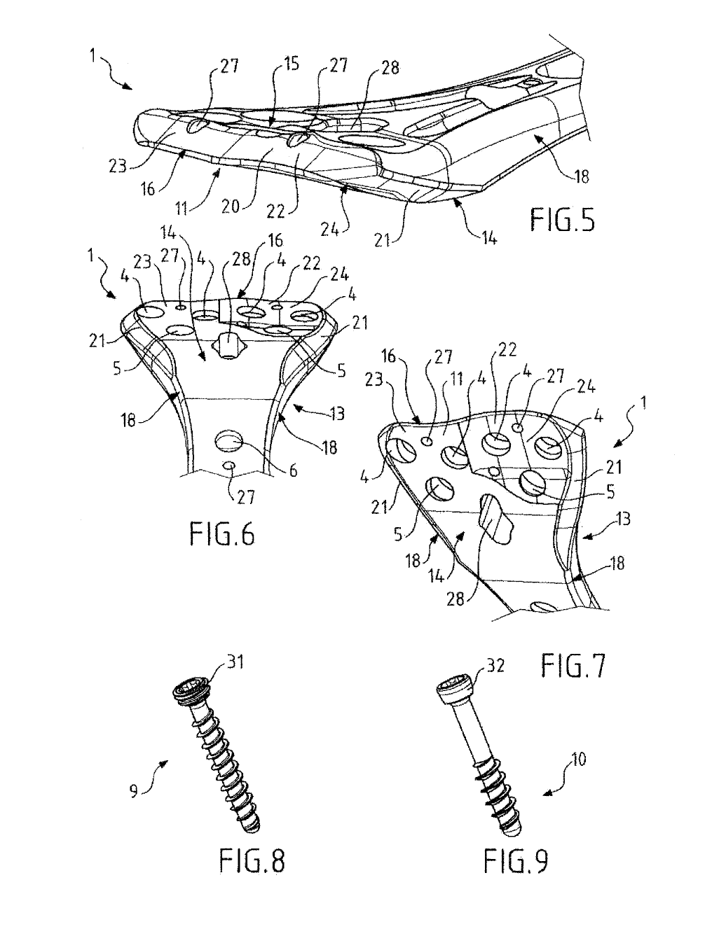

As illustrated in particular in FIGS. 6 and 7, the distal plate 11 preferably includes a concavity 24 arranged in the anterior surface 14 and extending from the ulnar portion 22 of said distal edge 16. The presence of this hollow shape which is the concavity 24 allows the main body to conform as well as possible to the physiological bone shape in order to limit the pain experienced by the patient and to adapt to the morphology of the latter.

Preferably, the main body 13 is provided with at least one longitudinal furrow, preferably two longitudinal furrows 25, 26, arranged at the bending area 19 of the main body 13, as shown for example in FIGS. 1 to 3 and 11. The longitudinal furrows 25, 26 are preferably disposed in a v-shape parallel to the lateral edges 18. Their longitudinal orientation allows them, while substantially preserving the mechanical properties in flexion of the main body linked to the thickness of the bending area 19 described previously, to lighten the main body 13 in order to facilitate its manufacture by molding. The longitudinal furrows 25, 26 are advantageously arranged in the posterior surface 15 of the main body 13 which is not intended to be in contact with the bone bodies 2, 3.

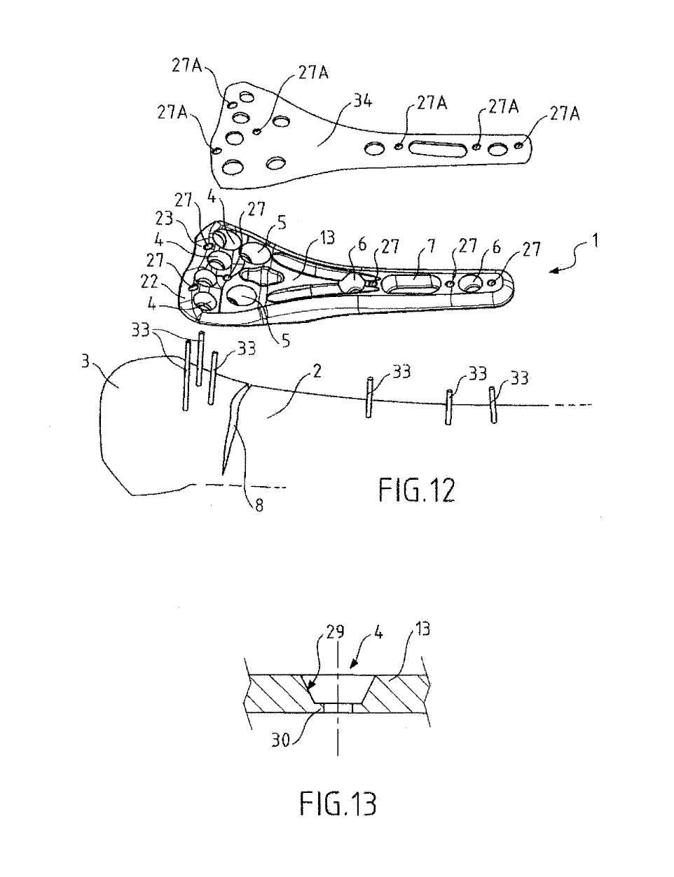

As illustrated in particular in FIG. 12, preferably, the surgical implant 1 comprises through guide holes 27 arranged in the main body 13, the surgical implant 1 being intended to be threaded via said guide holes 27 on guide pins 33 during its setting up in the patient's body. The guide pins 33 are, for example, implanted in the bone bodies 2, 3, prior to the setting up of the surgical implant 1 by itself, and intended to be removed after the setting up of said surgical implant 1. The setting up and the positioning of the surgical implant 1 is thus particularly easy and accurate.

The figures moreover illustrate a preferred example of embodiment of the fastening means 4, 5, 6, 7.

Preferably, the main body 13 is provided with at least one distal screw opening 4, 5 and at least one proximal screw opening 6, which are arranged respectively in the distal plate 11 and in the proximal tab 12, and which form or contribute to form the fastening means 4, 5, 6, 7.

The screw openings are preferably intended to receive, each, a fastening screw 9, 10 of the surgical implant 1 on the bone bodies 2, 3, and are oriented so as to induce a divergent positioning of said fastening screws 9, 10 in the main body 13, that is to say, cradle-like oriented, in order to ensure the reliability of the fastening and the holding of the surgical implant 1.

Preferably, at least three distal screw openings 4, preferably four, are arranged in the main body 13 and disposed at the same distance from each other of the distal edge 16, the distal screw openings 4 forming, or contributing to form, the fastening means 4, 5, 6, 7.

According to this preferable configuration, the distal screw openings 4 have a substantially transverse alignment and suitable for following the physiological curve of the bone body 3, in particular formed by the end of the radius, in which the corresponding fastening screws 9, 10 are intended to be implanted. Such an alignment allows the main body 13 to be slightly deformed by limiting the pain experienced by the patient, while ensuring an optimal holding of the implant on the bone body 3.

In the preferable variant illustrated in Figures, the main body 13 is provided with four distal screw openings 4 aligned along the distal edge 16, of two intermediate distal screw openings 5 distributed transversely in the distal plate 11 and away from the distal edge 16, near the through window 28. In this preferable variant, the main body 13 is also provided with two proximal screw openings 6, each of which being placed at one end of the proximal tab 12 so as to be longitudinally aligned.

As shown in Figures, the proximal tab 12 is provided with a longitudinal oblong screw opening 7 being intended to receive a fastening screw 9, 10 of the surgical implant 1 on the bone body 2, 3 while authorizing the sliding of said fastening screw 9, 10 along said oblong screw opening 7, in particular when said fastening screw 9, 10 is not or a little tight. Thanks to this preferred design, the surgeon can operate an adjustment to the proximity of the bone bodies 2, 3 when only the oblong screw opening 7 is occupied by a fastening screw 9 for the proximal tab 12 (The proximal screw openings 6 then being devoid of screws), by sliding said fastening screw 9, 10 in the oblong screw opening 7.

Such as illustrated in FIG. 13, at least one of the screw openings 4, 5, 6, 7, preferably all the screw openings 4, 5, 6, 7, advantageously forms a retention cone 29 of the screw via the head of the latter, the retention cone 29 being extended by an inner collar 30 forming a sub-head bearing surface of the fastening screw 9, 10. The axial retention cone 29 of the screw opening 4, 5, 6, 7, allows, during the tightening of the fastening screw 9, 10, to exert a radial pressure on the head 31, 32 of the latter, in order to substantially prevent any risk of unscrewing said fastening screw 9, 10, and any movement of the surgical implant 1 relative to the bone bodies 2, 3. The inner collar 30 in turn allows to give an orientation to the fastening screw 9, 10, in this case, so that the fastening screws 9, 10 are cradle-like oriented, divergently, when they are in place within the main body 13.

For example, for fastening we can use both:

at least one fastening screw 9 with threaded head 31 (as illustrated for example in FIG. 8), the threads of its threaded head 31 being intended to be screwed in the retention cone 29, and

at least one tightening fastening screw 10 with a non-threaded conical head 32 (as illustrated for example in FIG. 9), allowing to tighten solidly the surgical implant 1 against the bone bodies 2, 3 of the patient.

The fastening screws 9, 10 shown by way of example in the Figures have a nominal diameter of 2.7 mm, a cylindro-conical head, a rounded penetration end, and a star-like head cavity of the type <<Torx>>. Of course, other types of fastening screws may be used without departing from the scope of the invention.

The invention also concerns as such a surgical kit including a surgical implant 1 as described above, and also a shoe 34 (as illustrated for example in FIG. 12) complementary in shape to the distal plate 11, so as to be removably attachable on said distal plate 11, for example during the setting up of the surgical implant 1 in a patient's body.

The surgical kit also comprises advantageously the guide pins 33 described above, as well as the fastening screws 9, 10.

The shoe 34 is provided to be temporarily deposited, during the setting up of the surgical implant 1 in the patient's body, on the posterior surface 15 of the latter.

The shoe 34 is advantageously provided with guide holes 27A corresponding to the guide holes 27 of the surgical implant 1, so that the guide holes 27A can be threaded on the guide pins 33 by being superimposed on the guide holes 27 of the surgical implant.

In the same way, the shoe 34 has advantageously piercing openings 4A, 5A, 6A, 7A designed to be superimposed on and to correspond to the screw openings 4, 5, 6, 7 of the surgical implant 1 when the shoe 34 is attached on the latter.

In this way, when the surgical implant 1 is positioned with the help of the guide pins 33 on the bone bodies 2, 3 of the patient, it is possible to carry out a piercing or pre-piercing of the bone bodies 2, 3 with the purpose of inserting the fastening screws 9, 10. The pre-piercing or piercing being carried out through the piercing openings 4A, 5A, 6A, 7A, the integrity of the screw openings 4, 5, 6, 7 is preserved, and the operation of setting up the surgical implant is facilitated.

Moreover, the shoe 34 is preferably made of a radio-visible material, preferably a metallic material, for example stainless steel or titanium. Upon the setting up of the surgical implant 1, it is thus possible to know precisely the positioning of the latter in the patient's body despite its preferable radiolucency (especially when it is made of PEEK). Indeed, the shoe 34 being visible on the radiography and attached directly on said surgical implant 1, the surgeon can perfectly estimate the positioning of the surgical implant 1 with the help of a radiography.

The invention can finally concern, as such, a method for setting up a surgical implant 1 as described above, using a surgical kit described above.

The method for setting up the surgical implant advantageously comprises the following successive steps:

the patient's body is incised in order to be able to introduce the surgical implant 1 on the inside, at the bone bodies 2, 3 to be treated,

guide pins 33 are implanted in the bone bodies 2, 3, for example by impaction, and optionally with the help of the surgical implant 1 or the shoe 34, the guide pins 33 can be impacted in the bone bodies 2, 3 through the guide holes 27 and 27A of the latter,

the surgical implant 1 is positioned on the bone bodies 2, 3 using the guide pins 33, so that the anterior surface 14 rests at least partially on the bone bodies 2, 3, the distal plate 11 being in contact with an extreme part of the bone to be treated forming the bone body 3 and the proximal tab 12 being in contact with an intermediate or median part of the bone to be treated forming the bone body 2,

the shoe 34 is attached to the surgical implant 1,

a radiography is carried out to check the correct positioning of the radiolucent surgical implant 1 on the bone bodies, by visualizing the radio-visible shoe 34,

a pre-piercing of the bone bodies is carried out through all or part of the piercing openings 4A, 5A, 6A, 7A of the shoe 34,

the shoe 34 is removed,

a first tightening fastening screw 10 is inserted in one of the distal screw openings 4 of the distal plate 11, and a second tightening fastening screw 10 is inserted in the oblong screw opening 7 of the proximal tab 12,

before tightening said first and second tightening fastening screws 10, the proximity and orientation of the bone bodies 2, 3 are adjusted by sliding the second fastening screw 10 along the oblong screw opening 7,

the first and second tightening fastening screws 10 are tightened,

and then the fastening screws 9 with threaded head 31 are inserted in all or part of the remaining screw openings 4, 5, 6, in order to ensure a durable fastening of the surgical implant 1 in the patient's body,

as the insertion and tightening operations of the fastening screws 9, 10 are carried out, the guide pins 33 are removed from the bone bodies,

the incision of the patient's body is closed.

Of course, some steps can be reversed without departing from the scope of the invention.

In the end, the surgical implant 1 set up consequently in an easy, accurate and rapid manner allows a good quality and particularly rapid osteosynthesis of the bone bodies 2, 3, while limiting the pain experienced by the patient, whatever its morphology.

### POSSIBILITY OF INDUSTRIAL APPLICATION

The invention finds its industrial application in the design, production and implementation of surgical implants for osteosynthesis, in particular designed for the treatment of fractures or cracks of bone bodies, in particular the forearm, the tibia or the foot.

1. A surgical implant (1) intended to be attached on bone bodies (2, 3) of a patient, for example his forearm, via fastening means (4, 5, 6, 7) in order to hold in position said bone bodies (2, 3) relative to each other, said surgical implant (1) including a main body (13) formed by a distal plate (11) which is extended by a proximal tab (12), the distal plate (11) extending substantially according to a distal extension plane (Pd), the proximal tab (12) extending substantially along a proximal extension plane (Pp) intersecting the distal extension plane (Pd), the proximal tab (12) and the distal plate (11) being linked by a bending area (19), the surgical implant (1) being characterized in that the bending area (19) has the highest thickness in the main body (13).

2. The surgical implant (1) according to claim 1, characterized in that the bending area (19) has a thickness greater than the respective thicknesses of the distal plate (11) and of the proximal tab (12).

3. The surgical implant (1) according to claim 1, characterized in that the bending area (1.9) includes a maximum thickness (Emax), the thickness of the bending area (19) being decreasing from the maximum thickness (Emax), on the one hand, in the direction of the distal plate (11), and on the other hand, in the direction of the proximal tab (12).

4. The surgical implant (1) according to claim 1, characterized in that the distal plate (11) is inclined relative to the proximal tab (12), so that the distal extension plane (Pd) and the proximal extension plane (Pp) are intersecting each other so as to form an angle of elevation (α) being comprised between 10° and 80°, preferably between 20° and 30°, and even more preferably about 25°.

5. The surgical implant (1) according to claim 1, characterized in that the main body (13) is provided with at least one longitudinal furrow, preferably two longitudinal furrows (25, 26), arranged at the bending area (19) of the main body (13).

6. The surgical implant (1) according to claim 1, characterized in that the main body (13) has:
an anterior surface (14) with a convex general shape intended to rest at least partially on said bone bodies (2, 3), and
a posterior surface (15) with a concave general shape opposite to the anterior surface (14), and having a bend less significant than the posterior surface (15) so as to form a maximum thickness (Emax) between the proximal tab (12) and the distal plate (11), at the bending area (19).

7. The surgical implant (1) according to claim 6, characterized in that the main body (13) extends:
longitudinally between a distal edge (16) from which extends the distal plate (11), and a proximal edge (17) from which extends the proximal tab (12), and
transversely between two side edges (18).

8. The surgical implant (1) according to claim 7, characterized in that said distal edge (16) is beveled on the side of the posterior surface (15) so as to have a posterior fillet (20).

9. The surgical implant (1) according to claim 8, characterized in that one portion at least of the side edges (18) is beveled on the side of the anterior surface (14) from the distal edge (16) so as to form anterior fillets (21).

10. The surgical implant (1) according to claim 7, characterized in that the distal edge (16) has an ulnar portion (22) and a radial portion (23), the latter being recessed relative to said ulnar portion (22) so as to conform to the anatomical line of the bone bodies (2, 3), in particular a bone of the radius of the patient.

11. The surgical implant (1) according to claim 10, characterized in that the distal plate (11) has a concavity (24) arranged in the anterior surface (14) and extending from the ulnar portion (22) of said distal edge (16).

12. The surgical implant (1) according to claim 7, characterized in that at least three distal screw openings (4), preferably four, are arranged in the main body (13) and disposed at the same distance as each other from the distal edge (16), the distal screw openings (4) forming, or contributing to form, the fastening means (4, 5, 6, 7).

13. The surgical implant (1) according to claim 7, characterized in that the side edges (18) are substantially symmetrical relative to each other vis-à-vis a median plane of the main body (13).

14. The surgical implant according to claim 7, characterized in that the side edges (18) are curved and diverge from each other from the free end of the proximal tab (12) to the opposite free end of the distal plate (11), so that the proximal tab (12) is extended by the distal plate (11) by widening gradually.

15. The surgical implant (1) according to claim 1, characterized in that a through window (28) is arranged through the main body (13) at the bending area (19), in order to allow in particular the insertion of an osteoinductive material in the patient's body through said bending area (19) of the surgical implant (1), for example with the aim of repairing a fracture or a crack of the bone bodies (2, 3).

16. The surgical implant (1) according to claim 1, characterized in that the main body (13) is provided with at least one distal screw-in opening and at least one proximal screw opening (6), which are arranged respectively in the distal plate (11) and in the proximal tab (12), and which form or contribute to form the fastening means (4, 5, 6, 7), said screw openings being intended to receive, each, a fastening screw (9, 10) of the surgical implant (1) on the bone bodies (2, 3), the screw openings being oriented so as to induce a divergent positioning of said fastening screws (9, 10) in the main body (13).

17. The surgical implant (1) according to claim 16, characterized in that at least one of the screw openings (4, 5, 6, 7) forms a retention cone (29) of the screw via the head of the latter, the retention cone (29) extending by an inner collar forming a sub-head bearing surface of the fastening screw (9, 10).

18. The surgical implant (1) according to claim 1, characterized in that the proximal tab (12) is provided with a longitudinal oblong screw opening (7) being intended to receive a fastening screw (9, 10) of the surgical implant (1) on the bone body (2, 3) while authorizing the sliding of said fastening screw (9, 10) along said oblong screw opening (7).

19. The surgical implant (1) according to claim 1, characterized in that it comprises through guide holes (27) arranged in the main body (13), the surgical implant (1) being intended to be threaded via said guide holes (27) on guide pins (33) during its setting up in the patient's body.

20. The surgical implant (1) according to claim 1, characterized in that the main body (13) forms a one piece and unitary part made of a radiolucent material, preferably polyether ether ketone (PEEK), the latter being optionally loaded with carbon.

21. The surgical implant (1) according to claim 1, characterized in that it forms a forearm implant, said bone bodies (2, 3) being preferably formed by a bone of the cracked or fractured radius of the patient's forearm.

22. A surgical kit including a surgical implant (1) according to claim 1, and being characterized in that it also includes a shoe (34) complementary in shape to the distal plate (11), so as to be removably attachable on said distal plate (11), for example during the setting up of the surgical implant (1) in a patient's body.
